(19) United States
(12) Patent Application Publication
(10) Pub. No.: US 2024/0398605 A1
(43) Pub. Date: Dec. 5, 2024

(54) Hydrotherapy cup

(71) Applicant: Shenzhen Hijoy Technology Co., Ltd., Shenzhen (CN)

(72) Inventor: Zhubiao Shao, Shenzhen (CN)

(21) Appl. No.: 18/799,971

(22) Filed: Aug. 9, 2024

(51) Int. Cl. A61F 5/41; A61H 19/00

(52) U.S. Cl. CPC A61F 5/41; A61H 19/00; A61F 2005/412

(57) ABSTRACT

The utility model relates to the technical field of hydrotherapy cups, in particular to a hydrotherapy cup, which comprises a hydrotherapy cup shell, wherein a contact colloid is clamped at the bottom of the hydrotherapy cup shell, a power compartment is arranged at the upper end of the hydrotherapy cup shell, the hydrotherapy cup shell, the power compartment and the contact colloid form a cavity with holes at the bottom end, the holes are used for air and water circulation, and bubbles are generated by the air circulation in the water and then have a massage effect; the power compartment comprises an intake pipe and an exhaust pipe connected with the cavity, a fixed cover is clamped at the upper end of the hydrotherapy cup shell, the power compartment is located in the fixed cover, and a control panel is inlaid in the middle of the fixed cover; the utility model can increase the effects of massage and water bath, so that the user is more comfortable.

### TECHNICAL FIELD

The utility model relates to the technical field of hydrotherapy cups, in particular to a hydrotherapy cup.

### BACKGROUND ART

After the traditional physiotherapeutic instrument is sleeved, the silicone material below will stick close to the human organ, so that a sealing state is formed between the silicone material and the human organ. When the instrument operates, the instrument extracts air inside the silicone material, so that a negative pressure state is formed inside the silicone material, and the negative pressure can be controlled by adjusting the air pumping rate. Negative pressure environment can help the body organ recover. However, the physiotherapeutic instrument has a single experience feeling, and the negative pressure state is not conducive to the activity of the human organ, resulting in insufficient stimulation of the human organ and causing the human organ to be fatigued again.

### SUMMARY OF THE UTILITY MODEL

The utility model aims to provide a hydrotherapy cup to solve the problems raised in the background art.

In order to realize the above purposes, the utility model provides the following technical proposal: a hydrotherapy cup comprises a hydrotherapy cup shell, wherein a contact colloid is clamped at the bottom of the hydrotherapy cup shell, a power compartment is arranged at the upper end of the hydrotherapy cup shell, the hydrotherapy cup shell, the power compartment and the contact colloid form a cavity with holes at the bottom end, the holes are used for air and water circulation, bubbles are generated by the air circulation in the water and then have a massage effect, the power compartment comprises an intake pipe and an exhaust pipe connected with the cavity, a fixed cover is clamped at the upper end of the hydrotherapy cup shell, the power compartment is located in the fixed cover, a sealing ring is arranged at the connection of the power compartment and the fixed cover, a control panel is inlaid in the middle of the fixed cover, and a sealing ring is arranged at the connection of the power compartment and the hydrotherapy cup shell.

Preferably, the contact colloid is a silicone, ABS, PC or TPE colloid, which is an elastic structure.

Preferably, an integrally formed connecting pipe is arranged on the bottom side wall of the hydrotherapy cup shell and is externally connected with a control element to control the flow rate, and water or air enters the cavity through the connecting pipe when the interior of the cavity is in a negative pressure state.

Preferably, the negative pressure in the cavity is realized by the power compartment located at the top, a water pump and a battery pack are arranged in the power compartment, the control panel is electrically connected with the water pump and the battery pack, the water pump is connected with the exhaust pipe, the water pump pumps out through the exhaust pipe to achieve a negative pressure state.

Preferably, the control element comprises a hose connected with the connecting pipe, a limiting valve is arranged in the middle of the hose, and the hose is a plastic, silicone or rubber pipe.

Preferably, the limiting valve is composed of a clamping frame and an extrusion wheel, wherein the clamping frame is sleeved onto the hose, a guide groove is arranged in the middle of the clamping frame and is inclined, the extrusion wheel is located in the guide groove, and the lower end of the extrusion wheel is propped against the hose.

Preferably, the limiting valve is a knob type valve.

Compared with the prior art, the utility model has the following advantages: water is injected into the cavity through the connecting pipe, and then air is injected into the cavity through the connecting pipe to form bubbles, which can drive water inside the cavity to flow and then massage and bathe the human organ, making the users more comfortable and experience different feelings; the massage strength can be changed by changing the speed of air injection to adapt to the habits of different people.

### BRIEF DESCRIPTION OF THE DRAWINGS

FIG. 1 is the connection diagram for the hydrotherapy cup body and the hose.

FIG. 2 is the internal connection diagram for the power compartment.

FIG. 3 is the enlarged view of the point A.

Signs in the drawing: 1. Hydrotherapy cup shell, 2. Fixed cover, 3. Control panel, 4. Hose, 5. Limiting valve, 6. Connecting pipe, 7. Clamping frame, 8. Extrusion wheel, 9. Power compartment, 10. Intake pipe, 11. Exhaust pipe, 12. Battery pack, 13. Water pump.

### DETAILED DESCRIPTION OF THE UTILITY MODEL

In order to deepen the understanding and knowledge of the utility model, the technical proposal in the embodiments of the utility model will be clearly and completely described and introduced below in combination with the drawings to the embodiments of the utility model. Obviously, the embodiments are only part of the embodiments of the utility model, but not all the embodiments, not for restricting the embodiment in any form. Based on the embodiments of the utility model, all other embodiments obtained by ordinary technicians in the field without making creative labor falls within the scope of protection of the utility model.

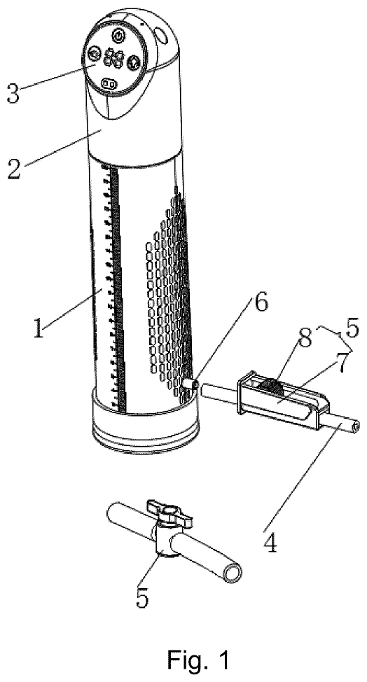

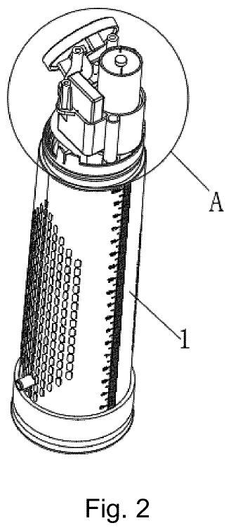

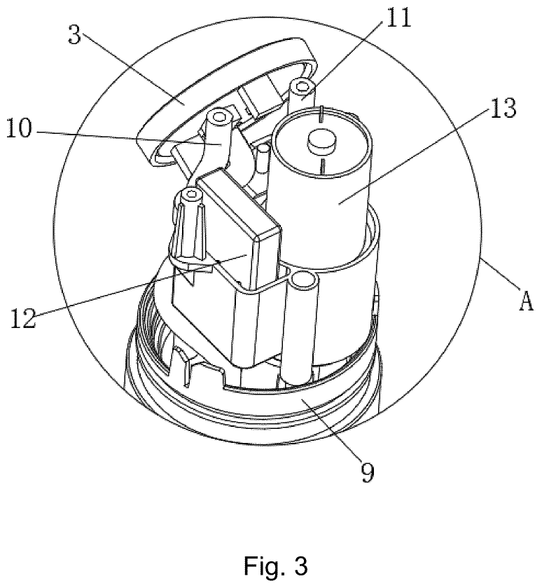

As shown in FIG. 1 to FIG. 3, utility model provides a technical proposal: a hydrotherapy cup comprises a hydrotherapy cup shell 1, a contact colloid is clamped at the bottom of the hydrotherapy cup shell 1, a power compartment 9 is arranged at the upper end of the hydrotherapy cup shell 1, the hydrotherapy cup shell 1, the power compartment 9 and the contact colloid form a cavity with holes at the bottom end, the holes are used for air and water circulation, and bubbles are generated by the air circulation in the water and then have a massage effect; the power compartment 9 comprises an intake pipe 10 and an exhaust pipe 11 connected with the cavity, a fixed cover 2 is clamped at the upper end of the hydrotherapy cup shell 1, the power compartment 9 is located in the fixed cover 2, and a sealing ring is arranged at the connection of the power compartment 9 and the fixed cover 2 to prevent water from entering the power compartment 9, resulting in damage to electronic devices; a control panel 3 is inlaid in the middle of the fixed cover 2, and a sealing ring is arranged at the connection of the power compartment 9 and the hydrotherapy cup shell 1 to improve the sealing between the power compartment 9 and the hydrotherapy cup shell 1, making the internal hydrotherapyce tighter and preventing air or water leakage caused by gaps; the water pump 13 in the power compartment 9 is controlled through the control panel 3 to inject water and air into the internal cavity.

The contact colloid is a silicone, ABS, PC or TPE colloid, which is an elastic structure; when being inserted into the human organ, the contact colloid can be deformed to be in contact with the human organ, and a sealing state is formed at the connection.

An integrally formed connecting pipe 6 is arranged on the bottom side wall of the hydrotherapy cup shell 1 and is externally connected with a control element 5 to control the flow rate, and water or air enters the cavity through the connecting pipe 6 when the interior of the cavity is in a negative pressure state; the negative pressure in the cavity is realized by the power compartment 9 located at the top, a water pump 13 and a battery pack 12 are arranged in the power compartment 9, the control panel 3 is electrically connected with the water pump 13 and the battery pack 12, the water pump 13 is connected with the exhaust pipe 11, and the water pump 13 pumps out through the exhaust pipe 11 to achieve a negative pressure state; the control element 5 comprises a hose 4 connected with the connecting pipe 6, a limiting valve 5 is arranged in the middle of the hose 4, and the hose 4 is a plastic, silicone or rubber pipe; when the hydrotherapy cup is used, the hose 4 can be inserted into the water first, and the water pump 13 can pump out the air inside the cavity, thus forming a negative pressure state, which can help the human organ erect, and the cavity can suck water under the negative pressure state, thus filling the cavity with water; then the excess water can be discharged from the exhaust pipe 11; after the cavity is filled with water, the hose 4 is drawn from the water; at this time the water pump can inject air into the cavity through the hose 4, and then bubbles can be formed in the cavity, which can drive the water inside the cavity to flow and then massage and bathe the human organ, thus achieving a more comfortable medicated bath effect.

The limiting valve 5 is composed of a clamping frame 7 and an extrusion wheel 8, wherein the clamping frame 7 is sleeved onto the hose 4, a guide groove is arranged in the middle of the clamping frame 7) and is inclined, the extrusion wheel 8 is located in the guide groove, and the lower end of the extrusion wheel 8 is propped against the hose 4; the limiting valve 5 is a knob type valve, which is an existing mature structure, and the internal structure is not shown in detail without affecting the integrity of the proposal; the speed of water and air flow into the cavity is controlled by controlling the limiting valve 5, thus giving the user a different experience.

Embodiment 1: The hydrotherapy cup of the utility model can be used as a traditional erection device. Before the operation of the equipment, the hose 4 is squeezed and sealed through the limiting valve 5. After the operation of the equipment, the water pump pumps out air in the internal cavity through the exhaust pipe, thus forming a negative pressure state in the internal cavity, promoting the erection of the human organ and changing the feeling of the human organ by changing the negative pressure;

Embodiment 2: On the basis of Embodiment 1, the limiting valve 5 is opened, and water is first injected into the internal cavity through the connecting pipe 6 until the internal cavity is filled with water. If water is continued to be sucked at this time, the water will be discharged from the exhaust pipe. When the internal cavity is filled with water, the gas is injected into the internal cavity through the hose, and then bubbles are formed to drive the water inside the cavity to flow and then massage and bathe the human organ, thus achieving a more comfortable medicated bath effect.

Although the embodiments of the utility model have been shown and described, it should be emphasized that the above description is only an introduction and description for the mode of use of the embodiments of the utility model, and is not a restriction on the utility model in any form. For ordinary technicians in the field, it can be understood that these embodiments may be changed, modified, substituted and varied without deviation from the principle and spirit of the utility model, and the scope of the utility model is limited by the claims and their equivalents.

1. A hydrotherapy cup, comprising a hydrotherapy cup shell (1), characterized in that a contact colloid is clamped at the bottom of the hydrotherapy cup shell (1), a power compartment (9) is arranged at the upper end of the hydrotherapy cup shell (1), the hydrotherapy cup shell (1), the power compartment (9) and the contact colloid form a cavity with holes at the bottom end, the holes are used for air and water circulation, bubbles are generated by the air circulation in the water and then have a massage effect, the power compartment (9) comprises an intake pipe (10) and an exhaust pipe (11) connected with the cavity, a fixed cover (2) is clamped at the upper end of the hydrotherapy cup shell (1), the power compartment (9) is located in the fixed cover (2), a sealing ring is arranged at the connection of the power compartment (9) and the fixed cover (2), a control panel (3) is inlaid in the middle of the fixed cover (2), and a sealing ring is arranged at the connection of the power compartment (9) and the hydrotherapy cup shell (1).

2. The hydrotherapy cup according to claim 1, characterized in that the contact colloid is a silicone, ABS, PC or TPE colloid, which is an elastic structure.

3. The hydrotherapy cup according to claim 1, characterized in that an integrally formed connecting pipe (6) is arranged on the bottom side wall of the hydrotherapy cup shell (1) and is externally connected with a control element (5) to control the flow rate, and water or air enters the cavity through the connecting pipe (6) when the interior of the cavity is in a negative pressure state.

4. The hydrotherapy cup according to claim 3, characterized in that the negative pressure in the cavity is realized by the power compartment (9) located at the top, a water pump (13) and a battery pack (12) are arranged in the power compartment (9), the control panel (3) is electrically connected with the water pump (13) and the battery pack (12), the water pump (13) is connected with the exhaust pipe (11), and the water pump (13) pumps out through the exhaust pipe (11) to achieve a negative pressure state.

5. The hydrotherapy cup according to claim 3, characterized in that the control element (5) comprises a hose (4) connected with the connecting pipe (6), a limiting valve (5) is arranged in the middle of the hose (4), and the hose (4) is a plastic, silicone or rubber pipe.

6. The hydrotherapy cup according to claim 5, characterized in that the limiting valve (5) is composed of a clamping frame (7) and an extrusion wheel (8), wherein the clamping frame (7) is sleeved onto the hose (4), a guide groove is arranged in the middle of the clamping frame (7) and is inclined, the extrusion wheel (8) is located in the guide groove, and the lower end of the extrusion wheel (8) is propped against the hose (4).

7. The hydrotherapy cup according to claim 5, characterized in that the limiting valve (5) is a knob type valve.
